(19) United States
(12) Patent Application Publication
(10) Pub. No.: US 2022/0189615 A1
(43) Pub. Date: Jun. 16, 2022

(54) DECENTRALIZED HEALTH MONITORING RELATED TASK GENERATION AND MANAGEMENT IN A HYPERCONVERGED INFRASTRUCTURE (HCI) ENVIRONMENT

(71) Applicant: VMware, Inc., Palo Alto, CA (US)

(72) Inventors: Xiang YU, Shanghai (CN); Yu WU, Shanghai (CN); Yang YANG, Shanghai (CN); Sifan LIU, Shanghai (CN); Jin FENG, Shanghai (CN); Xiaohua FAN, Shanghai (CN)

(73) Assignee: VMware, Inc., Palo Alto, CA (US)

(21) Appl. No.: 17/161,631

(22) Filed: Jan. 28, 2021

(30) Foreign Application Priority Data
Dec. 11, 2020 (CN) PCT/CN2020/135676

(51) Int. Cl. G16H 40/20; G16H 40/67

(52) U.S. Cl. CPC G16H 40/20; G16H 40/67

(57) ABSTRACT

A decentralized method for generation and management of health monitoring related tasks in a hyperconverged infrastructure (HCl) environment is provided. The hosts in the HCl environment each include a health agent and a task manager. The health agent collects health results from health checks and stores the health results in a shared database that is shared by the hosts. The task manager generates a health monitoring related task in response to the health results being indicative of a change in health status, and stores the health monitoring related task in a task pool that is also shared by the hosts. Any of the hosts can obtain and execute the health monitoring related tasks in the task pool based on a task priority and load balancing criteria.

### CROSS-REFERENCE TO RELATED APPLICATION

The present application claims the benefit of Patent Cooperation Treaty (PCT) Application No. PCT/CN2020/135676, filed Dec. 11, 2020, which is incorporated herein by reference.

### BACKGROUND

Unless otherwise indicated herein, the approaches described in this section are not admitted to be prior art by inclusion in this section

Virtualization allows the abstraction and pooling of hardware resources to support virtual machines in a software-defined networking (SDN) environment, such as a software-defined data center (SDDC). For example, through server virtualization, virtualized computing instances such as virtual machines (VMs) running different operating systems (OSs) may be supported by the same physical machine (e.g., referred to as a host). Each virtual machine is generally provisioned with virtual resources to run an operating system and applications. The virtual resources may include central processing unit (CPU) resources, memory resources, storage resources, network resources, etc.

A hyperconverged infrastructure (HCl) is one example implementation involving virtualization. A HCl is a software-defined framework that combines all of the elements of a traditional data center (e.g., storage, compute, networking, and management) into a unified system. With respect to storage functionality, a HCl may be used to create shared storage for VMs, thereby providing a distributed storage system in a virtualized computing environment. Such software-defined approach virtualizes the local physical storage resources of each of the hosts and turns the storage resources into pools of storage that can be divided and assigned to VMs and their applications. The distributed storage system typically involves an arrangement of virtual storage nodes into clusters wherein virtual storage nodes communicate data with each other and with other devices.

To effectively manage a large-scale distributed system, such as a distributed storage system, system administrators need to understand the current operational status of the system and need to take necessary actions against outages in the system. This is usually performed via continuous health monitoring of each host, along with a large amount of data aggregations and data analysis so as to get a cluster-level picture of the health of the system.

Typically, health check results (metrics) are collected from the hosts by a management server, and then aggregated and analyzed for diagnosis purposes and reported by the management server. The management server usually performs such health monitoring related tasks sequentially. This execution/processing of the health monitoring related tasks is performed sequentially by the management server due to at least two reasons: (1) there are health checks with dependencies, for example if the host is already down, there is no further need to check the host's disk health since a call to the host will be unsuccessful, and (2) the management server is a single node that may have limited resources.

Thus in view of at least the foregoing centralized arrangement wherein the management server performs the health monitoring related tasks, several drawbacks may result. One drawback is that there may be significant delay between when an abnormal event occurs and when the event is recognized as requiring the raising of a health alarm/notification. For instance, the management server (acting as a central node) may trigger health checks proactively with a relatively large time interval between sequential health checks (e.g., performs health checking every hour), and so some time may lapse before an anomalous health condition is detected by a regularly scheduled health check. Another drawback is that the management server can easily become a bottleneck, since the management server is a single node with limited resources and may be incapable of adequately and efficiently handling a large number of health monitoring related tasks when the clusters are scaled out significantly.

Furthermore in a HCl system, a cluster-wide view of the HCl system is needed in order to sufficiently detect and diagnose health problems. Health monitoring techniques that use distributed sensors to monitor the respective health of local hosts are inadequate for providing cluster-wide health assessment of an HCl system.

### BRIEF DESCRIPTION OF DRAWINGS

FIG. 1 is a schematic diagram illustrating an example virtualized computing environment having a distributed storage system and that implements a method to generate and manage health monitoring related tasks in a decentralized manner;

FIG. 2 is a schematic diagram illustrating further details of elements of the virtualized computing environment of FIG. 1 that are involved in decentralized generation and management of health monitoring related tasks;

FIG. 3 is a diagram of an example dependency tree of health results that may be used by the elements shown in FIG. 2;

FIG. 4 is a diagram showing a first example of decentralized generation and management of health monitoring related tasks that may be implemented based on the dependency tree in FIG. 3;

FIG. 5 is a diagram showing a second example of decentralized generation and management of health monitoring related tasks that may be implemented based on the dependency tree in FIG. 3;

FIG. 6 is a diagram showing a third example of decentralized generation and management of health monitoring related tasks that may be implemented based on the dependency tree in FIGS. 3; and

FIG. 7 is a flowchart of an example method to perform decentralized generation and management of health monitoring related tasks in the virtual computing environment of FIG. 1.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. The aspects of the present disclosure, as generally described herein, and illustrated in the drawings, can be arranged, substituted, combined, and designed in a wide variety of different configurations, all of which are explicitly contemplated herein.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, such feature, structure, or characteristic may be effected in connection with other embodiments whether or not explicitly described.

The present disclosure addresses the above-described drawbacks, by providing a distributed health check framework that meets demands for scalability, low latency for health checks, and more efficient consumption of sources in the hosts/HCl.

The health check framework performs decentralized data processing wherein cluster-wide health data processing tasks including aggregation and analysis can be executed by any node. Those tasks can be executed in parallel to reduce the latency, with the dependency managed. Further, the health check framework enables incremental system status updates along with the corresponding tasks being generated dynamically so as to avoid a global fresh to reduce the unnecessary resource consumption and to support reporting health status in real time. Also, the health check framework provides load balancing wherein the processing tasks are distributed among all nodes so as to avoid exhausting resources in a specific node and to reduce the latency.

Computing Environment

In some embodiments, the technology described herein may be implemented in a hyperconverged infrastructure (HCl) that includes a distributed storage system provided in a virtualized computing environment. In other embodiments, the technology may be implemented in other types of computing environments (which may not necessarily involve storage nodes in a virtualized computing environment). For the sake of illustration and explanation, the various embodiments will be described below in the context of a distributed storage system provided in a virtualized computing environment.

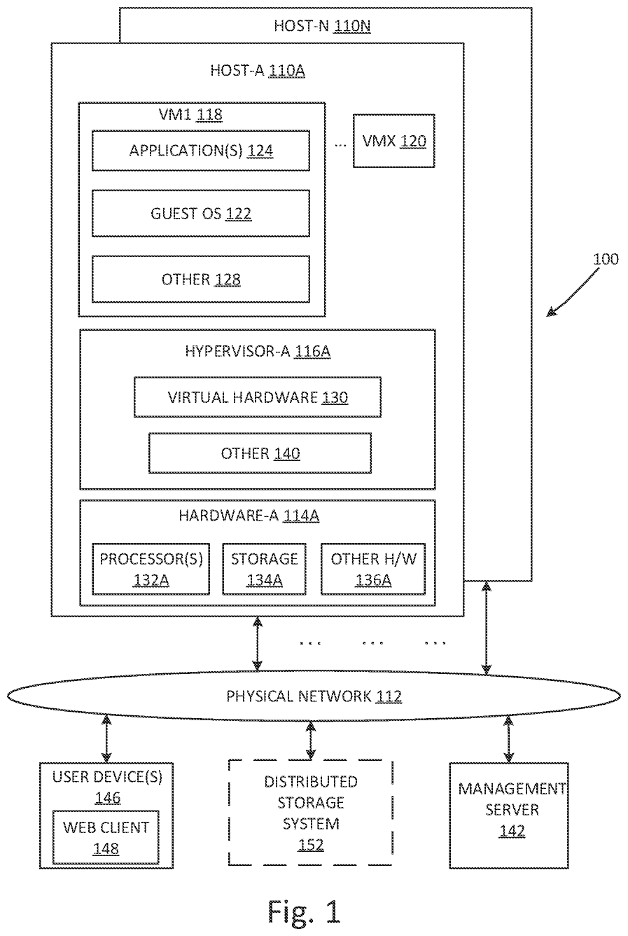

Various implementations will now be explained in more detail using FIG. 1, which is a schematic diagram illustrating an example virtualized computing environment 100 having a distributed storage system and that implements a method to generate and manage health monitoring related tasks in a decentralized manner. Depending on the desired implementation, virtualized computing environment 100 may include additional and/or alternative components than that shown in FIG. 1. The virtualized computing environment 100 can form part of a HCl framework in some embodiments.

In the example in FIG. 1, the virtualized computing environment 100 includes multiple hosts, such as host-A 110A . . . host-N 110N that may be inter-connected via a physical network 112, such as represented in FIG. 1 by interconnecting arrows between the physical network 112 and host-A 110A . . . host-N 110N. Examples of the physical network 112 can include a wired network, a wireless network, the Internet, or other network types and also combinations of different networks and network types. For simplicity of explanation, the various components and features of the hosts will be described hereinafter in the context of host-A 110A. Each of the other hosts can include substantially similar elements and features.

The host-A 110A includes suitable hardware-A 114A and virtualization software (e.g., hypervisor-A 116A) to support various virtual machines (VMs). For example, the host-A 110A supports VM1 118 . . . VMX 120. In practice, the virtualized computing environment 100 may include any number of hosts (also known as a “computing devices”, “host computers”, “host devices”, “physical servers”, “server systems”, “physical machines,” etc.), wherein each host may be supporting tens or hundreds of virtual machines. For the sake of simplicity, the details of only the single VM1 118 is shown and described herein.

VM1 118 may include a guest operating system (OS) 122 and one or more guest applications 124 (and their corresponding processes) that run on top of the guest operating system 122. VM1 118 may include still further other elements, generally depicted at 128, such as a virtual disk, agents, engines, modules, and/or other elements usable in connection with operating VM1 118.

The hypervisor-A 116A may be a software layer or component that supports the execution of multiple virtualized computing instances. The hypervisor-A 116A may run on top of a host operating system (not shown) of the host-A 110A or may run directly on hardware-A 114A. The hypervisor-A 116A maintains a mapping between underlying hardware-A 114A and virtual resources (depicted as virtual hardware 130) allocated to VM1 118 and the other VMs. The hypervisor-A 116A may include still further other elements, generally depicted at 140, such as a virtual switch, agent(s), etc. According to various embodiments that will be described later below, the other elements 140 may include a health agent and a task manager that cooperate with other elements in the virtualized computing environment 100 to provide decentralized generation and management of health monitoring related tasks.

Hardware-A 114A includes suitable physical components, such as CPU(s) or processor(s) 132A; storage resources(s) 134A; and other hardware 136A such as memory (e.g., random access memory used by the processors 132A), physical network interface controllers (NICs) to provide network connection, storage controller(s) to access the storage resources(s) 134A, etc. Virtual resources (e.g., the virtual hardware 130) are allocated to each virtual machine to support a guest operating system (OS) and application(s) in the virtual machine, such as the guest OS 122 and the applications 124 in VM1 118. Corresponding to the hardware-A 114A, the virtual hardware 130 may include a virtual CPU, a virtual memory, a virtual disk, a virtual network interface controller (VNIC), etc.

Storage resource(s) 134A may be any suitable physical storage device that is locally housed in or directly attached to host-A 110A, such as hard disk drive (HDD), solid-state drive (SSD), solid-state hybrid drive (SSHD), peripheral component interconnect (PCI) based flash storage, serial advanced technology attachment (SATA) storage, serial attached small computer system interface (SAS) storage, integrated drive electronics (IDE) disks, universal serial bus (USB) storage, etc. The corresponding storage controller may be any suitable controller, such as redundant array of independent disks (RAID) controller (e.g., RAID 1 configuration), etc.

A distributed storage system 152 may be connected to each of the host-A 110A . . . host-N 110N that belong to the same cluster of hosts. For example, the physical network 112 may support physical and logical/virtual connections between the host-A 110A . . . host-N 110N, such that their respective local storage resources (such as the storage resource(s) 134A of the host-A 110A and the corresponding storage resource(s) of each of the other hosts) can be aggregated together to form a shared pool of storage in the distributed storage system 152 that is accessible to and shared by each of the host-A 110A . . . host-N 110N, and such that virtual machines supported by these hosts may access the pool of storage to store data. In this manner, the distributed storage system 152 is shown in broken lines in FIG. 1, so as to symbolically convey that the distributed storage system 152 is formed as a virtual/logical arrangement of the physical storage devices (e.g., the storage resource(s) 134A of host-A 110A) located in the host-A 110A . . . host-N 110N. However, in addition to these storage resources, the distributed storage system 152 may also include stand-alone storage devices that may not necessarily be a part of or located in any particular host. The various storage resources in the distributed storage system 152 further may be arranged as storage nodes in a cluster.

A management server 142 or other management entity of one embodiment can take the form of a physical computer with functionality to manage or otherwise control the operation of host-A 110A . . . host-N 110N, including operations associated with the distributed storage system 152. In some embodiments, the functionality of the management server 142 can be implemented in a virtual appliance, for example in the form of a single-purpose VM that may be run on one of the hosts in a cluster or on a host that is not in the cluster of hosts. The management server 142 may be operable to collect usage data associated with the hosts and VMs, to configure and provision VMs, to activate or shut down VMs, to generate alarms and provide other information to a system administrator, and to perform other managerial tasks associated with the operation and use of the various elements in the virtualized computing environment 100 (including managing the operation of the distributed storage system 152). In one embodiment, the management server 142 may be configured to fetch health information from a shared database and to provide the health information to a system administrator via a user interface (UI), and to initiate a proactive user-triggered health check (which will be described later below).

The management server 142 may be a physical computer that provides a management console and other tools that are directly or remotely accessible to a system administrator or other user. The management server 142 may be communicatively coupled to host-A 110A . . . host-N 110N (and hence communicatively coupled to the virtual machines, hypervisors, hardware, distributed storage system 152, etc.) via the physical network 112. The host-A 110A . . . host-N 110N may in turn be configured as a datacenter that is also managed by the management server 142. In some embodiments, the functionality of the management server 142 may be implemented in any of host-A 110A . . . host-N 110N, instead of being provided as a separate standalone device such as depicted in FIG. 1.

A user may operate a user device 146 to access, via the physical network 112, the functionality of VM1 118 . . . VMX 120 (including operating the applications 124), using a web client 148 that provides a user interface. The user device 146 can be in the form of a computer, including desktop computers and portable computers (such as laptops and smart phones). In one embodiment, the user may be a system administrator that uses the web client 148 of the user device 146 to remotely communicate with the management server 142 via a management console for purposes of performing operations such as configuring, managing, diagnosing, remediating, etc. for the VMs and hosts (including triggering a proactive health check for the distributed storage system 152).

Depending on various implementations, one or more of the physical network 112, the management server 142, and the user device(s) 146 can comprise parts of the virtualized computing environment 100, or one or more of these elements can be external to the virtualized computing environment 100 and configured to be communicatively coupled to the virtualized computing environment 100.

Decentralized Generation and Management of Health Monitoring Related Tasks

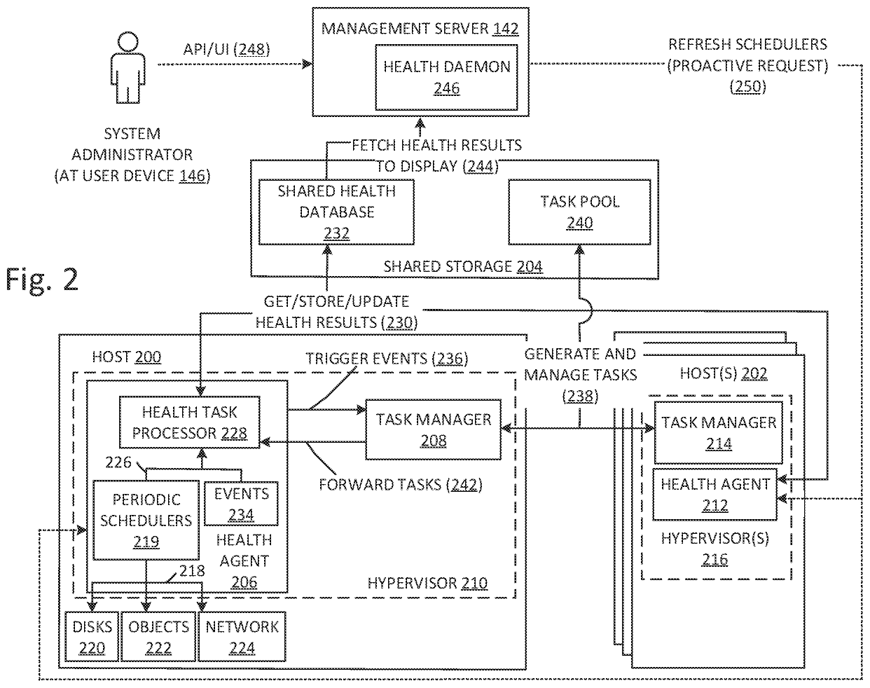

FIG. 2 is a schematic diagram illustrating further details of elements of the virtualized computing environment 100 of FIG. 1 that are involved in decentralized generation and management of health monitoring related tasks. Such elements include a host 200 and one or more other hosts 202 (which may be amongst the host-A 110A . . . host-N 110N in FIG. 1), a shared storage 204 (which may be one or more of the storage nodes in the distributed storage system 152 of FIG. 1 or may be located elsewhere in the virtualized computing environment 100), and the management server 142.

The host 200 includes a health agent 206 and a task manager 208. According to one embodiment, the health agent 206 and the task manager 208 may reside in or may be sub-elements of a hypervisor 210 that runs on the host 200. The host(s) 202 may each include a similar health agent 212 and task manager 214 that reside in or may be sub-elements of respective hypervisor(s) 216.

The health agent 206 locally monitors the health of the host 200 via health checks (shown at 218) issued by a periodic scheduler 219. For instance, the health agent 206 may monitor the health of disks 220, objects 222, network components 224, and various other elements of the host 220. The health checks may be triggered periodically, may be triggered based on certain conditions, and/or may be initiated/performed based on some other type of triggering/timing mechanism.

The results of these health checks are provided (shown at 226) to a health task processor 228 of the health agent 206. The health task processor 228 in turn provides (shown at 230) the results of the health check to a shared health database 232 (at the shared storage 204) for storage in the shared health database 232. If the result(s) of the health check(s) performed by the health agent 206 indicates a change or other type of event 234 (e.g., an outage or other change in health status/condition), the health task processor 228 (a) updates (shown at 230) the corresponding health results in the shared health database 232, and also (b) triggers the events (shown at 236) to the task manager 208 so that the task manager 208 may generate health monitoring related tasks to be stored (shown at 238) in a task pool 240 at the shared storage 204.

For example, a health check may detect an outage, which corresponds to an event that initiates one or more subsequent health monitoring related task. Such health monitoring related task(s), which the task manager 208 may generate and store in the task pool 240, may include various processing operations that pertain to the detected event, such as aggregation and analysis for diagnosis purposes, reporting to the management server 142, etc. As will be described later below, the task manager 208 may generate tasks for multiple levels of a dependency tree. For instance, if the results of the execution of the task at a particular level of the dependency tree indicates a change, then the task manager generates a next level of task processing from the dependency tree, and so forth until a root node is reached wherein further task execution is no longer needed.

The task manager of each host may manage/assign tasks from the task pool 240 to health agents, based on factors such as capacity of a particular host (its health agent) to execute the health monitoring related task, load balancing criteria (so as to avoid overloading a particular hosts and to reduce latency), priority of the health monitoring related task, task dependencies, etc. As depicted by way of example in FIG. 2, the task manager 208 at the host 200 may pull (shown at 238) a task from the task pool 240 and forward (shown at 242) the task to the health agent 206 for execution. In some embodiments, a task manager may assign tasks to its own host but not to other hosts, while in other embodiments, a task manager can assign tasks to its own host as well as to other hosts. The tasks may be executed in parallel, with managed dependencies. Further details regarding the generation and management of tasks by the task managers will be described later below. The health agent(s) assigned to execute the health monitoring related tasks can in turn obtain any health information (shown at 230) from the shared health database 232 that may be necessary to successfully complete the health monitoring related tasks (e.g., for aggregation, analysis, etc.).

FIG. 2 also shows (at 244) that a health daemon 246 may fetch health results from the shared health database 232 for display. For instance, a system administrator may operate a user interface at the user device 146 to display results of health checks, to view alarms, etc. Moreover, the user device 146 can generate an application program interface (API) call or other type of communication to instruct (shown at 248) the health daemon 246 at the management server 142 to refresh schedulers (shown at 250) after execution of health monitoring related tasks or to perform other proactive requests (including requests to perform health checks).

According to various embodiments, two types of workflows for health monitoring related tasks may be provided. One workflow involves automatically updating system health status and generating alarms to notify a system administrator when necessary, without requiring (or involving relatively minimal) user interaction. Another workflow is proactive in nature and is triggered by a system administrator to obtain the latest health information.

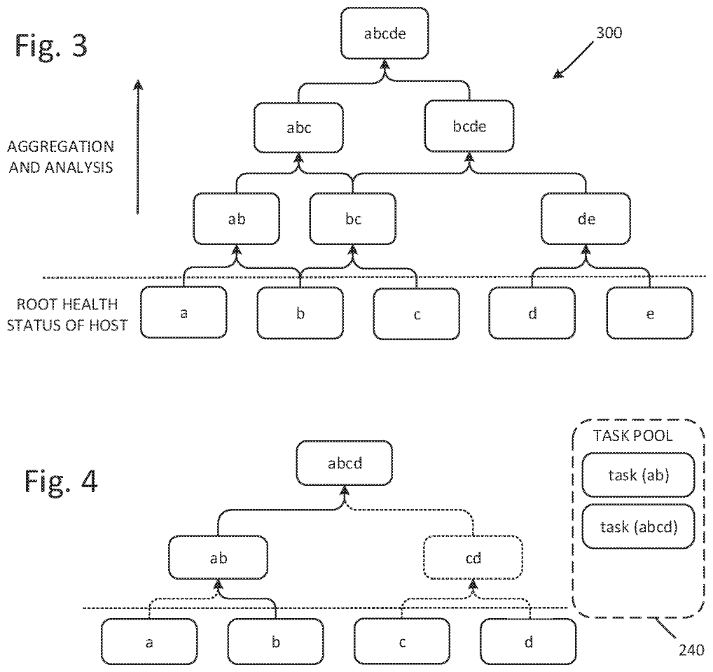

The automatic updating may be thought of as a bottom-up approach, and is depicted by way of example in FIG. 3. More specifically, FIG. 3 is a diagram of an example dependency tree 300 of health results that may be used by the elements (e.g., the task managers) shown in FIG. 2. A root health status of one or more hosts is depicted in the dependency tree 300 as a, b, c, d, and e. Each of a, b, c, d, and e may represent the health of a host itself and/or the health of a component of a host (such as a disk). Each health agent obtains health data to generate the leaf health result for a, b, c, d, and e. Above the root health status a, b, c, d, and e are one or more parent nodes which represent a cluster-wide health result with a corresponding health monitoring related task that can be placed in the task pool 240 and executed by any host at any appropriate time. For instance in FIG. 3, the parent node for a and b is ab; the parent node for b and c is bc; and the parent node for d and e is de. Still further, the parent node for ab and bc is abc; the parent node for bc and de is bcde; and the parent node for abc and bcde is abcde.

As shown in FIG. 3, there are dependencies between health results in different levels of the dependency tree 300. If the health result of one node is not changed, then the parent tasks (e.g., depicted in FIG. 3 as aggregation and analysis) do not need to be triggered since the upper health check status will not be changed. Therefore, the entire process of generating health results for the cluster looks like a bottom-up partial reconstruction of the dependency tree 300.

According to one embodiment, the dependency tree 300 may be programmed into each of the task managers shown in FIG. 2. The management server 142 may program the dependency tree 300 into the task managers, as well as updating the dependency tree as components are added to each host, clusters are scaled out, etc. In other embodiments, the task managers may access a dependency tree that is stored outside of the host(s).

FIG. 4 is a diagram showing a first example of decentralized generation and management of health monitoring related tasks that may be implemented based on the dependency tree 300 in FIG. 3. In this first example, the health check result indicates an update/change in the health status of b at the root leaf. Accordingly, a task manager (e.g., the task manager 208 in FIG. 2) generates/triggers a health monitoring related task ab for the parent node and places this task ab in the task pool 240. Any host (e.g., their respective task manager) can then pull/obtain the task ab from the task pool 240 for execution, based on certain factors/policies (described later below).

Based on the output of the task ab (e.g., updated information), the parent task abcd is triggered by the task manager and placed in the task pool 240. Again, any host (e.g., their respective task manager) can then pull the task abcd from the task pool 240 for execution, based on certain factors/policies (described later below).

As indicated in FIG. 4, the other paths/task cd is not activated/executed, since there was no update/change in the leaf health results c and d. Thus, avoiding the execution of task cd saves resources.

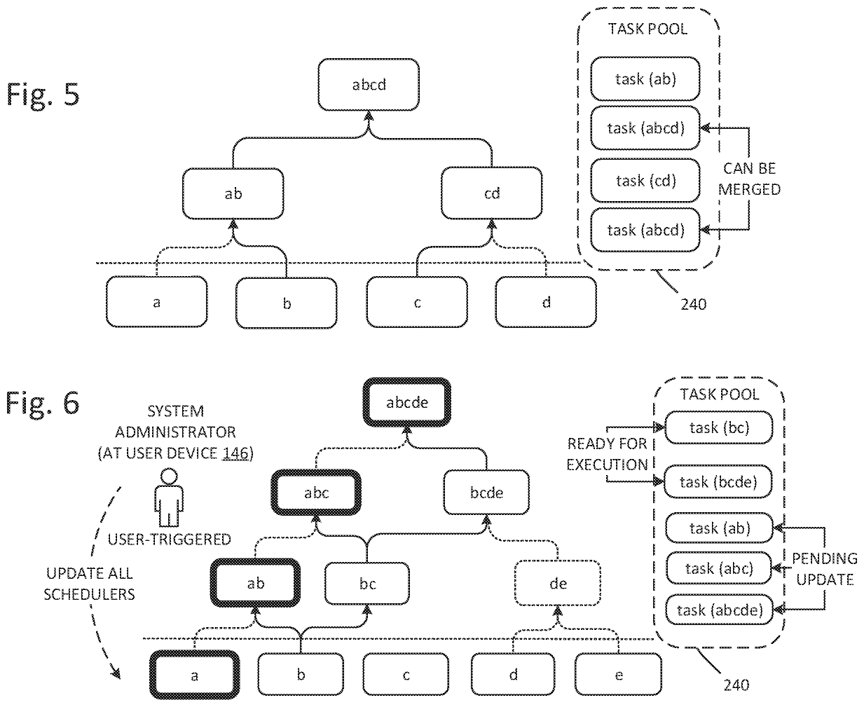

FIG. 5 is a diagram showing a second example of decentralized generation and management of health monitoring related tasks that may be implemented based on the dependency tree 300 in FIG. 3. In this second example, both leaf health result b and leaf health result c indicate updates/changes in health status, and so host b triggers task ab. In parallel, host c triggers task cd. Task ab and task cd are placed into the task pool 240, and then pulled and executed by one or more hosts.

The results of executing each of the tasks ab and cd triggers a task abcd. More specifically, task ab triggers task abcd, while task cd also triggers task abcd, from two different paths. Both of the triggered tasks abcd are placed in the task pool 240. If the first of these tasks in the task pool is not yet started, then the task manager can merge the two tasks abcd into a single task. For health result management of each task, a version control feature may be utilized to handle invalid tasks. For instance, the version control feature can generate identifiers, timestamps, etc. to identify valid/invalid and duplicate tasks.

Merging the same tasks can save system resources to avoid duplicated workload. In situations where merger is not possible or practical, the two tasks can be treated/executed independently. When the first task has been added to the task pool 240, that task can be executed first to return the health check result. This health check may not be truly up-to-date because the update from another path has not yet been executed/aggregated. However, such a condition may be tolerable because the health check result will be up-to-date once the second task is complete by following the same process. If the time difference between two same tasks is very small (e.g., in the order of milli-seconds), execution of both tasks may still be a waste of resources. Therefore, more policies may be defined to provide improvement in resource utilization. For example, the first task can wait a short time to see if there are any duplicated incoming tasks. The waiting time can be tuned for different scenarios. In one example implementation (for a top-down workflow described next), the parent health task can only be started when all child health results that it depends on have been updated, which can be judged through a refresh time.

FIG. 6 is a diagram showing a third example of decentralized generation and management of health monitoring related tasks that may be implemented based on the dependency tree 300 in FIG. 3. Specifically, FIG. 6 shows a proactive workflow that may be thought of as a top-down approach (a special case of the bottom-up approach described above) and that may be proactively triggered by the system administrator at the user device 146 via an API call.

In this third example, when the proactive request from the management server 142 is received by the hosts, the request time is recorded, and all bottom schedulers (e.g., the periodic schedulers 219 shown in FIG. 2) are refreshed immediately so as to enable the health agents at the hosts to update the latest leaf health check results. The parent tasks generated on-demand by the task schedulers into the task pool 240 can only be available to execute (e.g., served) when all child health results are ready, which means the oldest refresh time of all the child health results should be newer than the request time from the management server 142. One embodiment provides a mechanism to ensure that the timestamp can be passed up to the root node even if the health result itself does not change on each sub node.

In the third example of FIG. 6, the health nodes shown with an unweighted (non-thickened) border indicate that all of its child health results have been updated, and a health node shown with a weighted (thickened) border indicates that all of its child health results are not fully ready. Hence, the tasks in task pool 240 can be divided into two categories: ready for execution (e.g., tasks be and bcde) and pending update from its child nodes (e.g., tasks ab, abc, and abcde). In some embodiments, tasks created in the top-down process have a higher priority than tasks created in the bottom-up process, and so the results can be returned more expediently in the top-down process.

Therefore from the foregoing description, a health monitoring related task can comprise a task that generates a target health result from multiple source health results. For each health check result in the dependency tree 300, each result has at least one associated task. Each task may have the following metadata in order to support task execution:
Current health result(s): The output of the task execution.
Child health result(s): The input of the task execution.
Weight(s): Empirical workload of executing the task on a current node.
Weighted depth(s): The maximum total weight from a current health result to a root health result.
State(s): A task is in a pending state once generated and turns to a running state once executed by at least one host.

Once a health monitoring related task is created in the shared task pool 240, any host can pick up the task for execution at any appropriate time. Various embodiments may schedule multiple tasks in a decentralized and distributed cluster based on at least two aspects: task priority and task load balance.

Example execution priorities for health monitoring related tasks will now be described, with respect to bottom-up and top-down workflow scenarios explained above, wherein once a leaf health result changes, all associated upper health results need to be refreshed (a bottom-up scenario, which may be a default mode), and wherein a user requests an up-to-date health result through an explicit API call (a top-down scenario that will run until the overall health result is updated).

Beginning first with a bottom-up scenario, there may be two possible kinds of task priority settings:

(1) Execute tasks far away from root nodes in high priority, since doing so can decrease the total task effort as there will be more opportunities to merge duplicated tasks.
(2) Execute tasks close to root nodes in high priority, since doing so can reflect delta changes to root nodes as soon as possible.

If computing resources are sufficient, all tasks can run in parallel, and incremental changes can be quickly reflected in the root node. However, if computing resources are insufficient, it may be important to reduce total task efforts. Therefore, priority setting (1) may be preferable in some situations. Furthermore, in order to prevent tasks near the root nodes from becoming hungry, some embodiments utilize another factor: task duration in pending state, so as to increase the priority level and thereby shorten the time-to-completion of the task, in accordance with the task priority formula below for a bottom-up scenario:

P=D×Pr+Pd

wherein:

P: Task priority

D: Task weighted depth

Pr: Policy ratio, which should be a positive value

Pd: Task duration in pending state

In a top-down scenario, all periodical schedulers in all hosts will refresh health results. There may be a surge of leaf health result changes and consequent health tasks. The various embodiments focus on the execution of those tasks involved in the final health result requested by the system administrator, and all other tasks can be suspended for the time being.

Every non-leaf health result including a root health result is generated from a group of leaf health results. A base time of a leaf health result is its generation time, while a base time of a non-leaf health result is the earliest base time of its child health results. Thus, if a user requests a new health result at time T1, the user should expect the new health result with a base time newer than T1:

CurrentBaseTime=min{Children'sBaseTime}

The task priority formula for a top-down scenario may be set forth as follows:

P=D×IA

wherein:

P: Task priority

D: Task weighted depth

IA: Task involvement adjustment. This value represents whether this task is involved in requesting a new health result triggered by a user. IA=1, if the base time of the current health result is older than the user request time while the base time of all its child health checks are newer than user request time; otherwise, IA=0.

Hosts will not execute a task with priority P=0. Therefore, tasks involved in the top-down scenario are scheduled, while other tasks are suspended until the top-down scenario is complete.

Now moving on to load balancing considerations, it may be generally non-ideal for a host to pick up most tasks while other hosts are doing nothing, or for no host to pick up pending tasks for a long time. Therefore, one embodiment defines upper and lower bounds of a task number for each host, so as to achieve load balancing among the hosts:

MaxTasksPerHost=min{Mt, M/N×Hwr}

wherein:

Mt: Maximum thread number serving health tasks in a host.

M: Total number of tasks in the task pool 240.

N: Total number of active hosts.

Hwr: High watermark ratio, which is a percentage over average task number per host; the value of Hwr is between 1.0 and 2.0, for example: 1.1.

MinTasksPerHost=M/N×Lwr

wherein:

Lwr: Low watermark ratio, which is a percentage of overage task number per host; the value of Lwr is between 0.0 and 1.0, for example: 0.3.

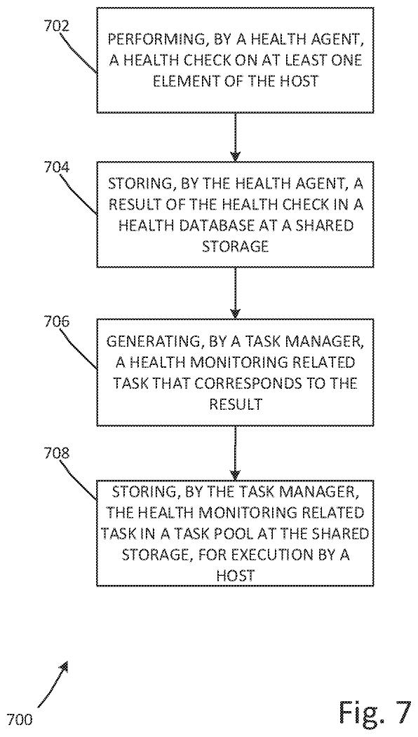

FIG. 7 is a flowchart of an example method 700 to perform decentralized generation and management of health monitoring related tasks in the virtual computing environment 100 of FIG. 1. The method 700 will further be described herein in the context of the elements shown in FIG. 2. The example method 700 may include one or more operations, functions, or actions illustrated by one or more blocks, such as blocks 702 to 708. The various blocks of the method 700 and/or of any other process(es) described herein may be combined into fewer blocks, divided into additional blocks, supplemented with further blocks, and/or eliminated based upon the desired implementation. In one embodiment, the operations of the method 700 and/or of any other process(es) described herein may be performed in a pipelined sequential manner. In other embodiments, some operations may be performed out-of-order, in parallel, etc.

The method 700 may begin at a block 702 (“PERFORMING, BY A HEALTH AGENT, A HEALTH CHECK ON AT LEAST ONE ELEMENT OF THE HOST”), wherein the health agent 206 at the host 200 (and/or the health agent 212 at any of the other hosts 202) performs a health check on various elements of the host, such as the disks 220, the objects 222, the network components 224, etc. These health checks generate health check results.

Next at a block 704 (“STORING, BY THE HEALTH AGENT, A RESULT OF THE HEALTH CHECK IN A HEALTH DATABASE AT A SHARED STORAGE”), the health agent 206 stores the health check results in the shared health database 232 at the shared storage 204. The health check results may indicate a change in health status of the element(s) of the host that were subject to a health check.

Hence at a block 706 (“GENERATING, BY A TASK MANAGER, A HEALTH MONITORING RELATED TASK THAT CORRESPONDS TO THE RESULT”), the task manager 208 generates a health monitoring related task that pertains to the result of the health check, and stores the health monitoring related task at the task pool 240 at a block 708 (“STORING, BY THE TASK MANAGER, THE HEALTH MONITORING RELATED TASK IN A TASK POOL AT THE SHARED STORAGE, FOR EXECUTION BY A HOST”). Once in the task pool 240, the health monitoring related task may be selected by any of the hosts for execution, based on factors such as load balancing criteria, task priority, task dependency, etc. as described previously above.

Computing Device

The above examples can be implemented by hardware (including hardware logic circuitry), software or firmware or a combination thereof. The above examples may be implemented by any suitable computing device, computer system, etc. The computing device may include processor(s), memory unit(s) and physical NIC(s) that may communicate with each other via a communication bus, etc. The computing device may include a non-transitory computer-readable medium having stored thereon instructions or program code that, in response to execution by the processor, cause the processor to perform processes described herein with reference to FIG. 2 to FIG. 7.

The techniques introduced above can be implemented in special-purpose hardwired circuitry, in software and/or firmware in conjunction with programmable circuitry, or in a combination thereof. Special-purpose hardwired circuitry may be in the form of, for example, one or more application-specific integrated circuits (ASICs), programmable logic devices (PLDs), field-programmable gate arrays (FPGAs), and others. The term “processor” is to be interpreted broadly to include a processing unit, ASIC, logic unit, or programmable gate array etc.

Although examples of the present disclosure refer to “virtual machines,” it should be understood that a virtual machine running within a host is merely one example of a “virtualized computing instance” or “workload.” A virtualized computing instance may represent an addressable data compute node or isolated user space instance. In practice, any suitable technology may be used to provide isolated user space instances, not just hardware virtualization. Other virtualized computing instances may include containers (e.g., running on top of a host operating system without the need for a hypervisor or separate operating system; or implemented as an operating system level virtualization), virtual private servers, client computers, etc. The virtual machines may also be complete computation environments, containing virtual equivalents of the hardware and system software components of a physical computing system. Moreover, some embodiments may be implemented in other types of computing environments (which may not necessarily involve a virtualized computing environment), wherein it would be beneficial to provide decentralized generation and management of health monitoring related tasks as described herein.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood that each function and/or operation within such block diagrams, flowcharts, or examples can be implemented, individually and/or collectively, by a wide range of hardware, software, firmware, or any combination thereof.

Some aspects of the embodiments disclosed herein, in whole or in part, can be equivalently implemented in integrated circuits, as one or more computer programs running on one or more computers (e.g., as one or more programs running on one or more computing systems), as one or more programs running on one or more processors (e.g., as one or more programs running on one or more microprocessors), as firmware, or as virtually any combination thereof, and that designing the circuitry and/or writing the code for the software and or firmware are possible in light of this disclosure.

Software and/or other computer-readable instruction to implement the techniques introduced here may be stored on a non-transitory computer-readable storage medium and may be executed by one or more general-purpose or special-purpose programmable microprocessors. A “computer-readable storage medium”, as the term is used herein, includes any mechanism that provides (i.e., stores and/or transmits) information in a form accessible by a machine (e.g., a computer, network device, personal digital assistant (PDA), mobile device, manufacturing tool, any device with a set of one or more processors, etc.). A computer-readable storage medium may include recordable/non recordable media (e.g., read-only memory (ROM), random access memory (RAM), magnetic disk or optical storage media, flash memory devices, etc.).

The drawings are only illustrations of an example, wherein the units or procedure shown in the drawings are not necessarily essential for implementing the present disclosure. The units in the device in the examples can be arranged in the device in the examples as described, or can be alternatively located in one or more devices different from that in the examples. The units in the examples described can be combined into one module or further divided into a plurality of sub-units.

We claim:

1. A method to perform decentralized generation and management of health monitoring related tasks in a virtual computing environment that includes multiple hosts arranged in a cluster, the method comprising:
performing, by a health agent at a host in the cluster, a health check on at least one element of the host;
storing, by the health agent, a result of the health check in a health database at a shared storage that is shared by the multiple hosts;
generating, by a task manager at the host in response to the result of the health check being indicative of a change in health status of the at least one element, a health monitoring related task that corresponds to the result; and
storing, by the task manager, the health monitoring related task in a task pool at the shared storage, wherein at least one host of the multiple hosts is configured to obtain the health monitoring related task from the shared storage for execution.

2. The method of claim 1, wherein the health monitoring related task includes a first health monitoring related task, and wherein the method further comprises:
generating, by the task manager, a second health monitoring related task that uses an output of the execution of the first health monitoring related task as an input and that is based on a task dependency tree; and
storing, by the task manager, the second health monitoring related task in the task pool at the shared storage, wherein the at least one of the multiple hosts is configured to obtain the second health monitoring related task from the shared storage for execution.

3. The method of claim 2, wherein the dependency tree includes a plurality of paths between parent nodes and child nodes, wherein the plurality of paths represent workflows for task execution, and wherein a first workflow for a first path of the plurality of paths is not executed if root nodes of the first workflow are not associated with a change in health status.

4. The method of claim 2, further comprising merging two tasks associated with at least two paths of the plurality of paths in the dependency tree, if the two tasks are same.

5. The method of claim 1, wherein selection of health monitoring related tasks from the task pool for execution by at least one host is based on a task priority or a load balancing criteria.

6. The method of claim 1, wherein selection of health monitoring related tasks from the task pool for execution by at least one host is based on a bottom-up approach, wherein the health monitoring related tasks are arranged in a dependency tree having upper and lower levels, and wherein execution of health monitoring related tasks at upper levels are started after health status changes at lower levels have been updated.

7. The method of claim 1, wherein selection of health monitoring related tasks from the task pool for execution by at least one host is based on a top-down approach, wherein the top-down approach is initiated in response to a request for health information received from a management server, wherein the health monitoring related tasks are arranged in a dependency tree having upper and lower levels, and wherein the request is served after health status changes at lower levels have been updated.

8. A non-transitory computer-readable medium having instructions stored thereon, which in response to execution by one or more processors, cause the one or more processors to perform or control performance of operations for decentralized generation and management of health monitoring related tasks in a virtual computing environment that includes multiple hosts arranged in a cluster, the operations comprising:
performing, by a health agent at a host in the cluster, a health check on at least one element of the host;
storing, by the health agent, a result of the health check in a health database at a shared storage that is shared by the multiple hosts;
generating, by a task manager at the host in response to the result of the health check being indicative of a change in health status of the at least one element, a health monitoring related task that corresponds to the result; and
storing, by the task manager, the health monitoring related task in a task pool at the shared storage, wherein at least one host of the multiple hosts is configured to obtain the health monitoring related task from the shared storage for execution.

9. The non-transitory computer-readable medium of claim 8, wherein the health monitoring related task includes a first health monitoring related task, and wherein the operations further comprise:
generating, by the task manager, a second health monitoring related task that uses an output of the execution of the first health monitoring related task as an input and that is based on a task dependency tree; and
storing, by the task manager, the second health monitoring related task in the task pool at the shared storage, wherein the at least one of the multiple hosts is configured to obtain the second health monitoring related task from the shared storage for execution.

10. The non-transitory computer-readable medium of claim 9, wherein the dependency tree includes a plurality of paths between parent nodes and child nodes, wherein the plurality of paths represent workflows for task execution, and wherein a first workflow for a first path of the plurality of paths is not executed if root nodes of the first workflow are not associated with a change in health status.

11. The non-transitory computer-readable medium of claim 9, wherein the operations further comprise:
merging two tasks associated with at least two paths of the plurality of paths in the dependency tree, if the two tasks are same.

12. The non-transitory computer-readable medium of claim 8, wherein selection of health monitoring related tasks from the task pool for execution by at least one host is based on a task priority or a load balancing criteria.

13. The non-transitory computer-readable medium of claim 8, wherein selection of health monitoring related tasks from the task pool for execution by at least one host is based on a bottom-up approach, wherein the health monitoring related tasks are arranged in a dependency tree having upper and lower levels, and wherein execution of health monitoring related tasks at upper levels are started after health status changes at lower levels have been updated.

14. The non-transitory computer-readable medium of claim 8, wherein selection of health monitoring related tasks from the task pool for execution by at least one host is based on a top-down approach, wherein the top-down approach is initiated in response to a request for health information received from a management server, wherein the health monitoring related tasks are arranged in a dependency tree having upper and lower levels, and wherein the request is served after health status changes at lower levels have been updated.

15. A system to perform decentralized generation and management of health monitoring related tasks in a virtual computing environment, the system comprising:
multiple hosts arranged in a cluster;
a shared storage that is shared by the multiple hosts; and
a health agent and a task manager at a host in the cluster, wherein:
the health agent is configured to perform a health check on at least one element of the host,
the health agent is configured to store a result of the health check in a health database at the shared storage,
the task manager is configured to generate, in response to the result of the health check being indicative of a change in health status of the at least one element, a health monitoring related task that corresponds to the result, and
the task manager is configured to store the health monitoring related task in a task pool at the shared storage, wherein at least one host of the multiple hosts is configured to obtain the health monitoring related task from the shared storage for execution.

16. The system of claim 15, wherein the health monitoring related task includes a first health monitoring related task, and wherein:
the task manager is configured to generate a second health monitoring related task that uses an output of the execution of the first health monitoring related task as an input and that is based on a task dependency tree, and
the task manager is configured to store the second health monitoring related task in the task pool at the shared storage, wherein the at least one of the multiple hosts is configured to obtain the second health monitoring related task from the shared storage for execution.

17. The system of claim 16, wherein the dependency tree includes a plurality of paths between parent nodes and child nodes, wherein the plurality of paths represent workflows for task execution, and wherein a first workflow for a first path of the plurality of paths is not executed if root nodes of the first workflow are not associated with a change in health status.

18. The system of claim 16, wherein two tasks associated with at least two paths of the plurality of paths in the dependency tree are merged, if the two tasks are same.

19. The system of claim 15, wherein selection of health monitoring related tasks from the task pool for execution by at least one host is based on a task priority or a load balancing criteria.

20. The system of claim 15, wherein election of health monitoring related tasks from the task pool for execution by at least one host is based on a bottom-up approach, wherein the health monitoring related tasks are arranged in a dependency tree having upper and lower levels, and wherein execution of health monitoring related tasks at upper levels are started after health status changes at lower levels have been updated.

21. The system of claim 15, wherein selection of health monitoring related tasks from the task pool for execution by at least one host is based on a top-down approach, wherein the top-down approach is initiated in response to a request for health information received from a management server, wherein the health monitoring related tasks are arranged in a dependency tree having upper and lower levels, and wherein the request is served after health status changes at lower levels have been updated.
